(19) United States
(12) Patent Application Publication
(10) Pub. No.: US 2002/0082635 A1
(43) Pub. Date: Jun. 27, 2002

(54) Conformal surgical balloon

(76) Inventors: Gene W. Kammerer, East Brunswick, NJ (US); Dorothy Dion, West Orange, NJ (US); Martin Nohilly, Hoboken, NJ (US)

Correspondence Address: Ralph W. Selitto, Jr., P.O. Box 1477, Edison, NJ, 08818-1477, US

(21) Appl. No.: 09/749,180

(22) Filed: Dec. 27, 2000

(51) Int. Cl. A61M 29/00

(52) U.S. Cl. 606/193

(57) ABSTRACT

A surgical balloon for insertion into a body cavity with a major chamber having a first volume and an annexed minor chamber communicating therewith and having a second volume less than the first volume has a continuous outer wall which defines the exterior surface of the balloon on a first side and the interior hollow of the balloon on a second side. The wall is formed from a stretchable elastic material permitting the interior hollow of the balloon to accommodate a variable volume of fluid ranging from a minimum deflated volume associated with a relaxed state of the wall to a maximum inflated volume associated with a stretched state of the wall. The wall provides the balloon with an inflated shape within the cavity approximating the interior shape of the body cavity, including a first portion approximating the major chamber and a second portion approximating the minor chamber. As applied to balloons for insertion into the uterus for endometrial ablation therapy, the balloon has or forms a pair of projections for inserting into the uterine cornua. The balloon may be preformed to mimic the uterine shape in the deflated state, may be formed from polyurethane and can have a varying wall expansibility and/or thickness promoting conformance of the inflated balloon to the uterine cavity.

### FIELD OF THE INVENTION

The present invention relates to surgical balloons, and more particularly to balloons suitable for introduction into a body cavity for containing a thermally conductive media used for ablation of cells within the cavity.

### BACKGROUND OF THE INVENTION

Surgical balloons have a variety of uses, including the containment of fluids used to necrose cells lining a body cavity. For example, it has now become common to treat excessive menstrual bleeding (menorrhagia) by inserting a balloon catheter into the uterus, filling the balloon with a thermally conductive media and heating or cooling the media to thermally kill the endometrial lining of the uterus. An exemplary thermal ablation process and apparatus utilizing a surgical balloon are described in U.S. Pat. No. 5,502,681 to Neuwirth et al.

As disclosed in U.S. Pat. No. 5,502,681, known surgical balloons are typically formed from latex, have a bulb shape, and inflate in a manner which enlarges the bulb shape uniformly to an approximately spherical or bulbous shape. In contrast, the uterine cavity is Y-shaped in cross-section. The material composition of known balloons is somewhat inelastic, preventing the balloons from readily conforming to the intra-uterine space. As a result, known bulbous surgical balloons do not inflate to contact the entire endometrial lining, in particular, in the area of the uterine cornua and, in some cases, adjacent the uterine os. This lack of contact may result in a portion of the endometrial lining escaping treatment.

It is therefore an object of the present invention to provide an improved surgical balloon that exhibits an increased contact area with a body cavity into which it is inserted when the balloon is inflated.

### SUMMARY OF THE INVENTION

The problems and disadvantages associated with the conventional surgical balloons are overcome by the present invention which includes a surgical balloon for insertion into a body cavity with a major chamber having a first volume and an annexed minor chamber communicating therewith and having a second volume less than the first volume. The balloon has a continuous outer wall which defines the exterior surface of the balloon on a first side and the interior hollow of the balloon on a second side. The wall is formed from a stretchable elastic material permitting the interior hollow of the balloon to accommodate a variable volume of fluid ranging from an minimum deflated volume associated with a relaxed state of the wall to a maximum inflated volume associated with a stretched state of the wall. The wall provides the balloon with an inflated shape within the cavity approximating the interior shape of the body cavity, including a first portion approximating the major chamber and a second portion approximating the minor chamber.

### BRIEF DESCRIPTION OF THE FIGURES

For a better understanding of the present invention, reference is made to the following detailed description of an exemplary embodiment considered in conjunction with the accompanying drawings, in which:

FIG. 1 is a plan view of an inflated surgical balloon in accordance with a first exemplary embodiment of the present invention;

FIG. 2 is a plan view of the balloon of FIG. 1, partially deflated;

FIG. 3 is a side view of a cannula partially sectioned to show the balloon of FIG. 1 deflated and stored within a cannula;

FIGS. 4a-4c are cross-sectional views of the balloon of FIG. 3 shown in three different folded configurations, taken along section line IV-IV and looking in the direction of the arrows;

FIG. 5 is a partial cross-sectional view of the surgical balloon of FIG. 1 folded in an alternative manner;

FIG. 6 is a plan view of a surgical balloon in accordance with an alternative exemplary embodiment of the present invention;

FIG. 7 is a plan view of a surgical balloon in accordance with another alternative embodiment of the present invention; and

FIG. 8 is a diagrammatic view of the balloon of FIG. 7 at three stages of inflation within a uterine cavity.

### DETAILED DESCRIPTION OF THE FIGURES

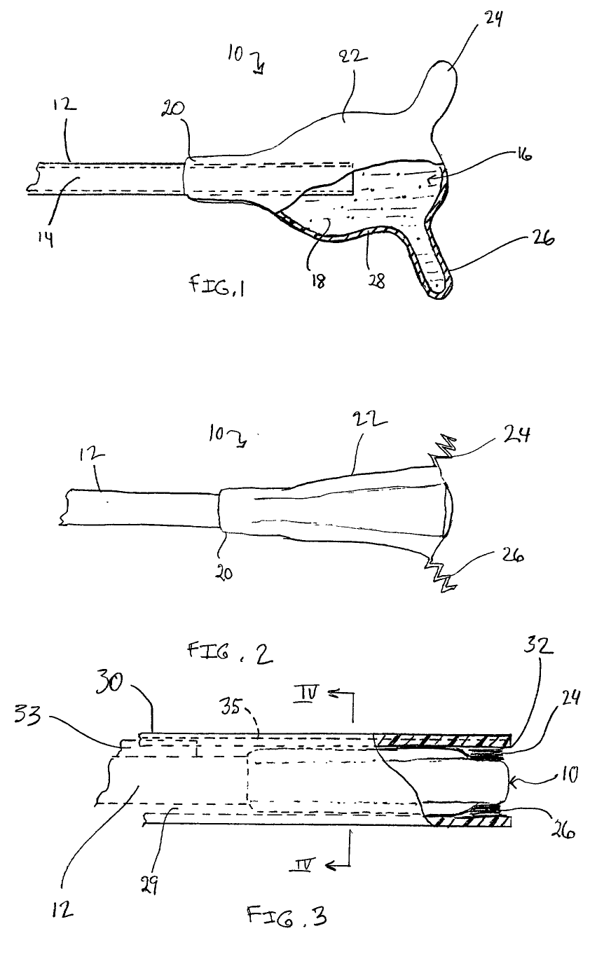

FIG. 1 shows a surgical balloon 10 disposed on the end of a catheter 12. The catheter 12 has a lumen 14 that communicates with an interior hollow 16 of the balloon 12 and permits the infusion of a thermally conductive fluid 18 into the balloon 10 under pressure. As is known in the art, surgical balloons may be used to perform surgical procedures, such as endometrial ablation to cure menorrhagia. U.S. Pat. No. 5,954,714, which is incorporated herein by reference, teaches the use of surgical balloons for endometrial ablation.

The balloon 10 can be used in place of conventional bulb-shaped balloons to perform ablation procedures. More particularly, after the balloon 10 has been introduced into the uterus, the pressurized thermally conductive fluid 18, e.g., saline solution, dextrose, etc., is used to inflate the balloon 10 within the uterus, followed by heating or cooling of the fluid to thermally cauterize cells in contact with the balloon 10.

The balloon 10 is preferably preformed to have a specific shape, such that when the balloon is inflated, it conforms to the walls of the intra-uterine space. The balloon 10 has a base 20 that is adhered to the catheter 12 by an adhesive or by plastic welding. The body 22 of the balloon 10 extends from the base 20 and has left and right extensions 24, 26. As can be appreciated from FIG. 1, the outer three-dimensional shape of the balloon 10 mimics the interior hollow of a uterus when the balloon 10 is inflated. In this manner, the balloon 10 can more completely fill the hollow of the uterus into which it is inserted and contact a greater surface area relative to a bulb-shaped balloon of the prior art. More particularly, the body 22 may extend from the isthmus to the fundus with the left and right extensions 24, 26 inserting into the uterine cornua. The greater contact area that may be achieved with the balloon 10 provides for greater thermal transfer and more complete endometrial ablation. Because the balloon 10 readily assumes a complementary shape relative to the body cavity into which it is placed, the balloon 10 conforms to the cavity shape without exerting as much pressure on the body cavity as the prior art balloons. In addition, an even pressure is exerted by the balloon 10 across the internal surface of the uterus promoting consistent and even contact therebetween which translates to a uniform ablation depth of the cells.

FIG. 1 shows a wall portion 28 of the balloon 10 which has a substantially uniform thickness. The balloon 10 may be blow molded from polyester or polyethylene resins; dip molded from silicones, natural, latex rubber or polyisoprene, a synthetic rubber; extrusion molded from silicone; injection molded from polyurethanes or silicones; or formed by heat sealing sections (patterns) together. Currently, the preferred method of manufacture is dip molding using natural, latex rubber or polyisoprene. Other compounds from which the balloon 10 can be made using one or more of the foregoing processes are polyether block amides, polyolefins and co-polyesters.

As referred to above, the balloon 10 can be formed by “heat sealing”, viz., by cutting patterns of a sealable expandable material into specific shapes and then heat sealing the edges of the two identical shapes together. This can produce a structure, which has a volume between the top and bottom patterns. An alternate method is to place two sheets of material together, one on top of the other. A formed die is then placed on either side of the two sheets, and the die is heated to melt the sections of the sheets between the corresponding sections of the die. This area creates a seal when it cools. The shape or configuration of the die determines the shape of the balloon 10.

The balloon 10 can be used in hot ablation procedures and in cryoablation. Materials, which are best suited for hot ablation procedures, would include polyisoprene, silicone and natural, latex rubber. The material best suited for use in cold ablation procedures would be silicone.

Polyurethane, in contrast to the latex compounds that have previously been used to make surgical balloons, is highly elastic and permits the balloon 10 to conform readily to the intrauterine space, even with minimal or no preforming. Accordingly, the present invention is intended to include the use of polyurethane to produce a bulb-shaped surgical balloon that conforms to the intrauterine shape to insure low pressure conformation to the intrauterine shape. For example, a bulbous polyurethane balloon is sufficiently elastic to conform to the uterine cavity so as to assume a shape approximating that shown in FIG. 1, when inflated within the uterus.

FIG. 2 illustrates the preformed balloon 10 of FIG. 1 after it has been partially deflated and the left and right extensions 24, 26 folded in a zig-zag pleated fashion to facilitate packing the balloon 10 in an introducer tube or cannula 30.

As can be appreciated from FIG. 3, the folded left and right extensions 24, 26 permit the extensions 24, 26 to be neatly stored along with the remainder of the balloon 10 within the lumen 29 of the cannula or introducer tube 30 that is used to facilitate introduction and deployment of the catheter 12 and balloon 10 into the uterus of a patient. More particularly, the cannula 30 can be slipped through the uterine os, followed by urging of the catheter 12 forward to deploy the balloon 10 beyond an open tip 32 of the cannula 30. In the alternative, the cannula 30 can be withdrawn backward off of the balloon 10, exposing it in place. Once the balloon 10 is unconstrained by the cannula 30, it expands and unfolds under the influence of its elastic memory. Unfolding is completed by the infusion of thermally conductive fluid into the balloon 10, inflating it to the shape shown in FIG. 1. Preferably, the balloon 10 is stored in a folded configuration that provides for a sequenced unfolding that properly positions the balloon 10 within the uterus to facilitate optimal balloon-to-endometrial lining contact when the thermally conductive media is infused into the balloon 10 inflating it and filling the intra-uterine space. The cannula 30 and/or the catheter 12 are therefore preferably provided with an orientation marking that allows the surgeon to insert the balloon 10 in the proper orientation relative to the patient. To maintain the relative position of the catheter 12 and the cannula 30, it is preferred that they be keyed relative to the other, e.g., that the catheter 12 be provided with a longitudinal ridge 33 that fits within a mating guide way 35 in the cannula 30. In this manner, the orientation of the balloon 10 is preserved and the balloon 10 is rotationally fixed relative to the cannula 30, thereby avoiding inadvertently disturbing the folded position of the balloon 10.

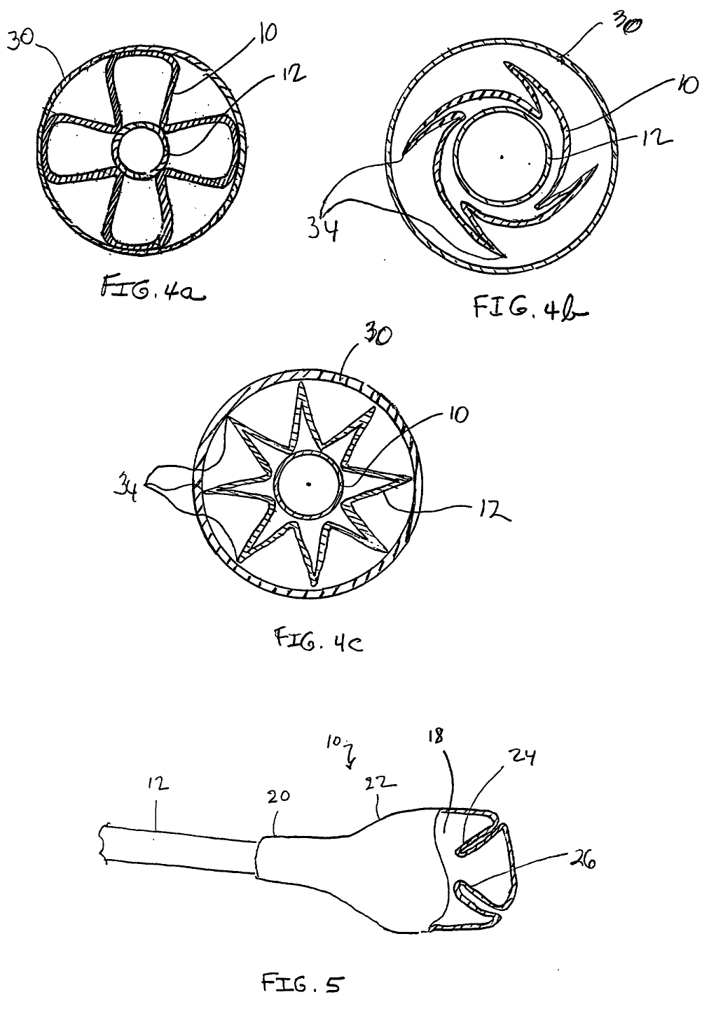

FIG. 4a shows how the balloon 10 is folded to allow storage in the cannula and to facilitate a controlled unfolding and deployment when the balloon 10 is pushed out of the cannula 30 into the uterus. When applied in the treatment of a body cavity having a directional sense, such as the uterus, the balloon 10 has a shape that requires orientation relative to the specific orientation of the body cavity in which it is deployed.

FIGS. 4b and 4c illustrate alternative configurations for folding balloon 10, i.e., in a pin-wheel and star shape, respectively. In accordance with the teachings of the present invention, the balloon 10 may be preformed to have an uninflated configuration as shown in any of FIGS. 4a-4c, in particular, the balloon 10 may be preformed to have sharp fold lines 34 without exceeding the elastic limit of the material of composition.

The selection of material for the balloon 10 insures that the balloon 10 will not stick to itself or the cannula 30 after prolonged storage within the cannula 30. Alternatively, the balloon 10 may be coated with a conventional, biocompatible, non-allergenic lubricant, such as corn starch, talc or low viscosity silicone, preferably air cured, to prevent self-adhesion. In order to promote deployment of the balloon 10, it is preferably folded in a manner that minimizes overlap, severity of fold angle and compression forces that exceed the elastic limit of the material at the fold lines. In addition, it is preferable that the extensions 24, 26 be folded at intervals that are smaller in length than the spacing between any opposed surfaces within the uterine cavity that could trap the extensions 24, 26 in an unfolded condition, e.g., between the walls of the uterine cornua or between the body 22 of the balloon 10 and the uterine wall.

FIG. 5 shows another method for folding the extensions 24, 26 for storage within the cannula 30. More particularly, the extensions 24, 26 can be introverted such that they project inwardly into the interior hollow 18 of the body 22 of the balloon 10.

FIG. 6 shows a balloon 110 in accordance with the present invention and having an outwardly flaring body 122, the upper peripheral edge 123 of which is intended to extend into the uterine cornua of a patient. The body 122 is axially symmetric, i.e., generally bell-shaped and therefore does not have discernable extensions 24, 26 in the uninflated state such as the embodiment of FIG. 1. When inflated, however, the overall shape of the balloon and corresponding distribution of flexible wall material allows the upper peripheral edge 123 to expand out into the cornua. The body 122 flares out rapidly from the base 120 to a lower circumference 121 to increase contact with the uterus proximate the uterine os. This effect is illustrated in FIG. 8 and described below.

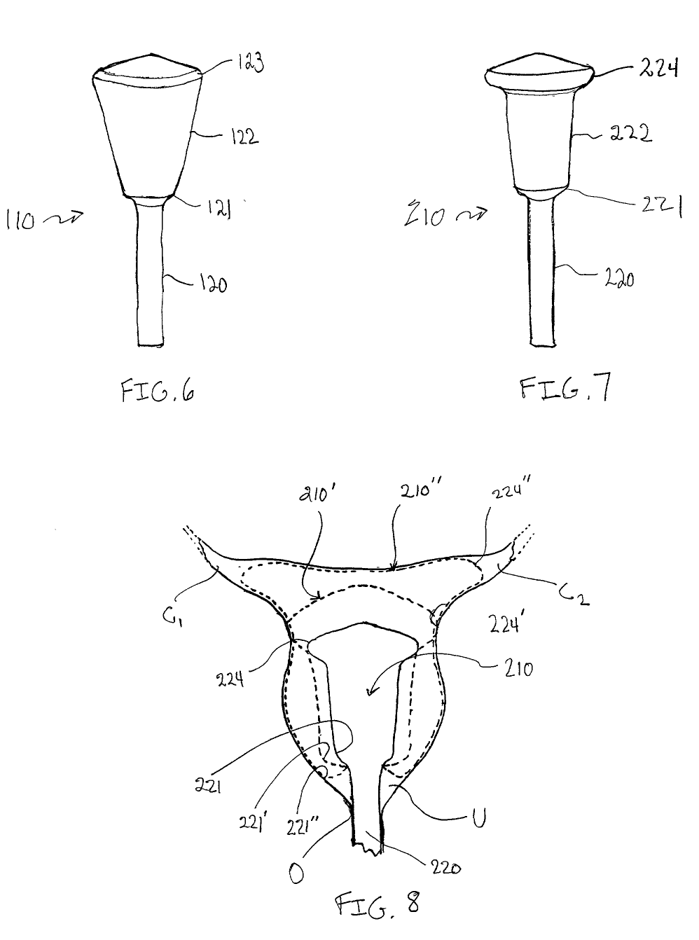

FIG. 7 shows a balloon 210 having a similar overall shape as the balloon 110 of FIG. 6, but with an expandable upper flange 224 to facilitate expansion into the uterine cornua. FIG. 8 illustrates the balloon 210 within a uterine cavity U, in the uninflated state, in an intermediate state of inflation 210′ (dotted) and inflated almost completely 210″ (dotted). As the balloon 210 is inflated, the expansible flange 224 (224′, 224″) approaches and enters the uterine cornua C1, C2. Simultaneously, lower circumference 221, (221′, 221″) immediately expands outwardly to contact the uterine cavity U proximate the os O. Depending upon the shape of the uterine cavity U, the lower circumference 221″ may project downwardly to fill the space between the base 220 and the uterine cavity U proximate the os O. Alternatively, the expansion of the balloon 210 will push the base 220 in an outward direction (i.e., to a lesser degree of insertion) such that the balloon 210 will establish maximum contact with the uterine cavity U. In either case, the “bell”-shaped balloons 110 and 210 establish greater contact with the uterine cavity U more quickly and completely than a conventional bulb-shaped balloon. Since the balloons 10, 110 and 210 more readily inflate to a shape approximating the cavity into which they are inserted, greater contact area at a more even, higher pressure is achieved, assuring better thermal transfer.

A commonly owned copending application Ser. No. ______ entitled “CONFORMAL SURGICAL BALLOON WITH VARYING WALL EXPANSIBILITY” and filed contemporaneously herewith by the present inventors, discloses a surgical balloon that conforms to the intra-uterine space aided by variations in wall expansibility due to varying wall thickness or varying elasticity, such copending application being incorporated herein by reference. The present invention therefore contemplates a preformed surgical balloon (mimicking the uterine cavity shape), or a bulb-shaped polyurethane balloon, wherein the wall thickness and/or elasticity varies in accordance with the teachings of the referenced co-pending application, to aid in permitting the balloon to conform to the body cavity in which it is inflated.

It should be understood that the embodiments described herein are merely exemplary and that a person skilled in the art may make many variations and modifications without departing from the spirit and scope of the invention as defined in the appended claims. All such variations and modifications are intended to be included within the scope of the present invention as defined in the appended claims.

I/We claim:

1. A surgical balloon for insertion into a body cavity with a major chamber having a first volume and an annexed minor chamber communicating therewith and having a second volume less than the first volume, comprising:
a continuous outer wall, said wall defining an exterior surface of the balloon on a first side and an interior hollow of the balloon on a second side, said wall being formed from a stretchable elastic material permitting said interior hollow of said balloon to accommodate a variable volume of fluid ranging from a minimum deflated volume associated with a relaxed state of said wall to a maximum inflated volume associated with a stretched state of said wall, said wall providing said balloon with an inflated shape within the body cavity approximating the interior shape of such cavity, including a first portion approximating the major chamber and a second portion approximating the minor chamber.

2. The surgical balloon of claim 1, wherein the body cavity is the uterus, the major chamber being the primary uterine space extending between the isthmus and fundus, the minor chamber being two in number and corresponding to the uterine cornua, and wherein the second portion of the wall approximates one uterine cornua and a third portion of the wall approximates the other cornua.

3. The surgical balloon of claim 2, wherein said wall is formed in a predetermined shape such that said balloon has an uninflated shape which approximates said inflated shape.

4. The surgical balloon of claim 3, further including a catheter having a lumen for conducting a fluid therethrough, said balloon sealingly attached to said catheter at one end thereof with said lumen in communication with the interior hollow of said balloon.

5. The surgical balloon of claim 4, further including a cannula to aid in introducing said balloon into the uterus, said balloon and said catheter slidably and removably contained within said cannula.

6. The surgical balloon of claim 5, wherein said balloon has a folded portion that is accommodated within said cannula, said folded portion having a generally cruciform cross-section.

7. The surgical balloon of claim 5, wherein said balloon has a folded portion that is accommodated within said cannula, said folded portion having a generally star-shaped cross-section.

8. The surgical balloon of claim 5, wherein said balloon has a folded portion that is accommodated within said cannula, said folded portion having a generally pin-wheel shaped cross-section.

9. The surgical balloon of claim 5, wherein said second portion and said third portion of said wall are flattened and folded in a zig-zag pattern when stored in said cannula.

10. The surgical balloon of claim 5, wherein said second portion and said third portion of said wall are introverted when stored in said cannula.

11. The surgical balloon of claim 5, wherein said cannula has an orientation marking thereon to permit a user thereof to ascertain the orientation of said balloon contained therein and alignment means for maintaining said cannula and said catheter in relative alignment when said catheter is stored within said cannula.

12. The surgical balloon of claim 3, wherein said wall is a material selected from the group consisting of polyurethane, latex, polyester, polyethylene, silicone and polyisoprene.

13. The surgical balloon of claim 3, wherein said wall has a substantially uniform thickness in the relaxed state.

14. The surgical balloon of claim 3, wherein said wall has a varying expansibility.

15. The surgical balloon of claim 14, wherein said varying expansibility is attributable to varying wall thickness.

16. The surgical balloon of claim 2, wherein said balloon is radially symmetric when uninflated.

17. The surgical balloon of claim 16, wherein said balloon is approximately bell-shaped.

18. The surgical balloon of claim 17, wherein said balloon has an expandible upper flange from which said second portion and said third portion are formed.

19. The surgical balloon of claim 17, wherein said wall has a varying expansibility.

20. The surgical balloon of claim 16, further including a catheter having a lumen for conducting a fluid therethrough, said balloon sealingly attached to said catheter at a base thereof with said lumen in communication with the interior hollow of said balloon, and wherein said balloon flares outwardly from said base to a first diameter greater than the diameter of said base to facilitate said balloon approximating said major chamber proximate an os of the uterus into which said balloon is introduced.

21. The surgical balloon of claim 4, wherein said balloon flares outwardly from said catheter to a first diameter to facilitate said balloon approximating said major chamber proximate an os of the uterus into which said balloon is introduced.

22. A method for making a surgical balloon for insertion into a body cavity with a major chamber having a first volume and an annexed minor chamber communicating therewith and having a second volume less than the first volume, comprising the steps of:
forming a continuous outer wall, said wall defining the exterior surface of the balloon on a first side and the interior hollow of the balloon on a second side, said wall being formed from a stretchable elastic material permitting the interior hollow of the balloon to accommodate a variable volume of fluid ranging from an minimum deflated volume associated with a relaxed state of said wall to a maximum inflated volume associated with a stretched state of said wall, said wall providing the balloon with an inflated shape within said cavity approximating the interior shape of the body cavity, said step of forming including forming a first portion of said wall having an inflated shape which approximates the major chamber and forming a second portion of said wall having an inflated shape which approximates the minor chamber.

23. The method of claim 22, wherein the body cavity is the uterus, the major chamber being the primary uterine space extending between the isthmus and the fundus, the minor chamber being two in number and corresponding to the uterine cornua and wherein said second portion approximates one uterine cornua and a third portion of said wall approximates the other cornua, said step of forming including forming said third portion of said wall.

24. The method of claim 23, wherein said step of forming is by dip molding.

25. The method of claim 23, wherein said step of forming is by blow molding.

26. The method of claim 23, wherein said step of forming is by extrusion.

27. The method of claim 23, wherein said step of forming is by heat sealing.

28. The method of claim 23, wherein the primary uterine space includes the space between the uterine os and the fundus and wherein said step of forming said first portion includes forming an outwardly directed radial flare terminating in a first diameter at one end of said first portion, said one end approximating the primary uterine space proximate the uterine os when said balloon is inflated.
